(19) United States
(12) Patent Application Publication
(10) Pub. No.: US 2022/0371987 A1
(43) Pub. Date: Nov. 24, 2022

(54) PROCESS FOR MANUFACTURE OF AMANTADINE NITRATE DERIVATIVES

(71) Applicant: QINGDAO HAILAN PHARMACEUTICALS CO., LTD., Qingdao (CN)

(72) Inventors: Yuqiang WANG, Qingdao (CN); Zheng LIU, Qingdao (CN); Yewei SUN, Qingdao (CN); Zaijun ZHANG, Qingdao (CN); Gaoxiao ZHANG, Qingdao (CN)

(73) Assignee: QINGDAO HAILAN PHARMACEUTICALS CO., LTD., Qingdao (CN)

(21) Appl. No.: 17/300,107

(22) PCT Filed: Sep. 11, 2019
(86) PCT No.: PCT/CN2019/000178

§ 371 Date: Mar. 11, 2021

(51) Int. Cl. C07C 231/06

(52) U.S. Cl. CPC C07C 231/06; C07C 2603/74

(57) ABSTRACT

The present invention provided a process for manufacture of amantadine nitrate derivatives, and the process comprises using adamantane as the raw material to prepare amantadine nitrate derivatives via the following steps: (1) synthesis of adamantanol; (2) carboxylation of adamantanol; (3) acetylation of adamantanoic acid; (4) reduction; (5) hydrolysis of amido adamantanol and Boc protection of amino group; (6) crystallization of Boc protected amantadinol; (7) nitrate esterification of Boc protected amantadinol; (8) refining of the product of nitrate esterification; (9) Boc deprotection and salt formation; and (10) refining of amantadine nitrate hydrochloride. The amantadine nitrate derivatives have the struction of:

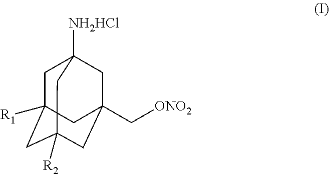

wherein, R₁ and R₂ are each independently hydrogen, straight-chain or branched-chain alkyl, or substituted or unsubstituted aryl or heteroaryl. The process of this invention is efficient, cost effective, environmentally friendly, safe, reliable, and suitable for industrial production.

### FIELD OF THE INVENTION

The present invention relates the medical field, and relates to a process for the manufacture of medicaments, and more particularly to a process for the manufacture of amantadine nitrate derivatives.

### BACKGROUND OF THE INVENTION

Amantadine and its derivatives have varieties of biological activities and are widely used in the field of medicine. Memantine (1,3-Dimethylamantadine, Memantine) is a non-competitive antagonist of NMDA receptors, and is mainly used for the treatment of moderate to severe Alzheimer's Disease (AD). By binding to the NMDA receptor in ion channel, memantine can block influx of K⁺ and Ca²⁺ plasmas with neuroprotective effects. The binding of memantine to the NMDA receptor is reversible with a moderate dissociation rate, which can ensure the pharmacological effects and also provent from being accumulated in the channel to affect the normal physiological functions of the receptor (Lipton et al., Journal of neurochemistry. 2006, 97: 1611-1626). Also, memantine, which binding to the NMDA receptor is voltage-dependent, can only bind to the receptor when the neuron is depolarized, and thus can block the activation of the NMDA receptor when the neuron continues to be depolarized under pathological conditions, but would not block the activation of NMDA receptor under normal physiological conditions (Wenk et al., CNS drug reviews. 2003, 9(3): 275-308; McKeage., Drugs & Aging. 2010, 27(2): 177-179).

Nitric oxide (NO) is indicated to have varieties of in vivo biological activities. As a free radical gas, NO has an unpaired electron with extremely unstable chemical properties and is very easy to combine with free radicals, and thereby reduces the number of free radicals and reduces the harm caused by oxidative damage to body tissues. In addition, as a signaling molecule, NO also plays multiple roles in the cardiovascular system. Endogenous NO is a vasodilator, which acts on guanylate cyclase in vascular smooth muscle cells to promote vasodilation and lower blood pressure. Also, it can enter platelet cells, reduce their activity, thereby inhibit their aggregation and adhesion to the vascular endothelium, prevent thrombosis, and prevent atherosclerosis. Small molecule drugs that can release NO, such as nitroglycerin, sodium nitroprusside, isosorbide mononitrate, are widely used for the treatment of many clinical diseases.

Amantadine nitrate compound (II) is a NO donating small molecule compound (CN105294450) independently designed and developed by the inventors of this patent application:

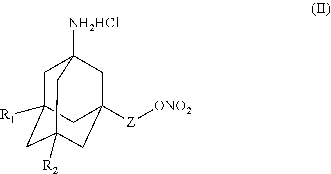

wherein R₁ and R₂ are each independently hydrogen, straight-chain or branched-chain alkyl, substituted or unsubstituted aryl or heteroaryl, substituted or unsubstituted esters, substituted amines; and Z straight- or branched-carbon chain with 0 to 6 carbon atoms connecting to the nitrate ester group and can be substituted with a heteroatom, alkyl group, aryl group, and aryl hetero group. Preferably, the alkyl group is a C1-C10 alkyl group.

Preliminary pharmacological studies have found that these compounds can play a similar role to memantine in effectively antagonizing the excessive activation of NMDA receptors caused by glutamate, inhibiting the influx of calcium ions, and scavenging free radicals in the cerebral cortex, and thereby effectively protect cerebral cortex neurons, and, on the other hand, it can release NO efficiently and relax arteries (Liu et al., Med. Chem. Comm., 2017, 8, 135-147).

Such compounds have shown good therapeutic effects in rat models for various diseases of nervous system or neurovascular system. Among them, the compound MN-08 (III) can significantly reduce the cerebral infarction area and cerebral edema in rats caused by ischemic stroke; significantly improve the memory impairment and behavioral impairment of vascular dementia rats; and also reduce monocrotaline-induced pulmonary artery pressure in rats with pulmonary hypertension, and inhibit pulmonary artery remodeling and right ventricular hypertrophy. In addition, MN-08 is indicated to have a good effect on rats with subarachnoid hemorrhage, glaucoma and other disease models.

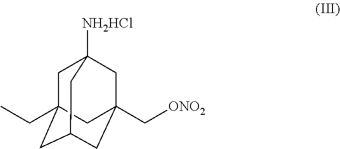

Therefore, as a new candidate class one drug molecule with completely independent intellectual property rights, the MN-08 and amantadine nitrate derivatives have broad development prospects and great practical and economic significance.

In the early stage, the inventors of the subject application explored a laboratory small-scale synthetic method of these compounds (CN105294450). In the method, substituted or unsubstituted adamantane was used as the raw material, which was brominated and hydrolyzed to obtain an adamantane alcohol. The adamantane alcohol undergoes Ritter reaction to give adamantanoic acid, and further undergoes Koch-Haff reaction to give acetamidoadamantic acid. In the compound the carboxyl group is reduced to obtain an acetamidoadamantane methanol. To the acetamidoadamantane methanol with diethylene glycol (15-20 Vol) as the solvent was added a high dose of sodium hydroxide for hydrolysis at high temperature. The hydrolyzed product with tetrahydrofuran as solvent was condensed with Boc anhydride under conditions of triethylamine and DMAP. The condensation product was nitrated with a nitrating reagent mixed with fuming nitric acid and acetic anhydride, and finally the Boc group was removed with HCl to obtain amantadine nitrate compound as shown in the following scheme.

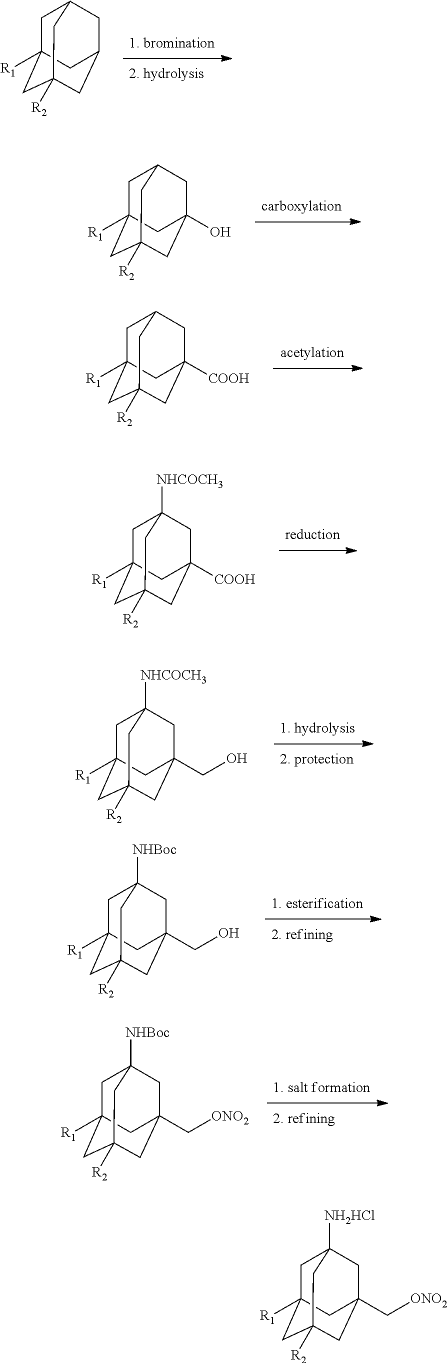

wherein R₁ and R₂ are each independently hydrogen, straight-chain or branched-chain alkyl, substituted or unsubstituted aryl or heteroaryl.

In the above synthetic method, the dosage ratio of the reagents and solvents is not optimized. The usage amount of the reaction reagents such as bromine, sodium hydroxide, and strong acid and strong base, such as sulfuric acid and nitric acid, is too high, which caused waste in reagents, increased costs, and added burden on environmental protection; moreover, the purification of the compounds after each step of the reaction was obtained through separation and purification with silica gel column, which is not suitable for industrial batch production.

More importantly, during the research process of the present invention, the inventors conducted research and development of a scale-up pilot process described herein below, and discovered the above-mentioned limitations of the previous laboratory small-scale synthesis method. The inventors discovered through scale-up pilot tests of ethyl-adamantane nitrate that, in the nitration reaction, when the reaction was run by using the relative amount of the materials in the laboratory small scale process, with the nitrifying agent fuming nitric acid and acetic anhydride being in the ratio of Starting Materials:Ac₂O:HNO₃=1:1.8:2.8, the reaction speed was found very fast, and the reactants were easily reacted into completion and quickly turned into side products (III), mostly being transformed into side products (1.5 h) within the reaction time, and subsequently the yield of nitrate esters was reduced from 60-70% to about 18%, which ultimately resulted in failure of the production process with impossible separation and purification of nitrate ester intermediates. Therefore, in the previous process, the usage amount of the nitrating reagent is not suitable for industrial production.

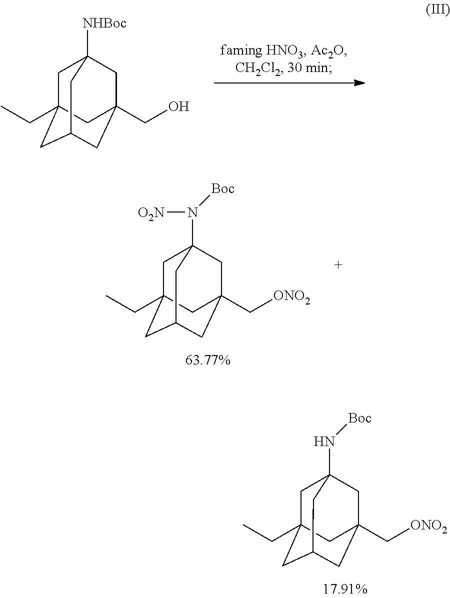

### SUMMARY OF THE INVENTION

The present invention is directed to solve the technical problems in a previous process and achieve optimization of the process conditions with significant reduction in the usage of various reaction reagents such as strong acids and strong bases, energy consumption, and costs of production; and also to provide an optimized post-treatment process suitable for industrial production; and further, more importantly, to adjust the amount of fuming nitric acid and acetic anhydride in the nitrating reagent, to find the appropriate process dosage and reaction rate to meet the requirements of production process, and to reduce the generation of by-products with simplified post-treatment process.

The purpose of the present invention is to provide a cost effective, green, safe and reliable industrial process for manufacture of amantadine nitrate derivatives.

The process for manufacture of amantadine nitrate derivatives of the present invention includes the following steps: (1) synthesis of adamantanol from adamantane; (2) carboxylation of adamantanol; (3) acetylation of adamantanoic acid; (4) reduction; (5) hydrolysis of amido adamantanol and Boc protection of amino group; (6) crystallization of Boc protected amantadinol; (7) nitrate esterification of Boc protected amantadinol; (8) refining of product of nitrate esterification; (9) Boc deprotection and salt formation; and (10) refining of amantadine nitrate hydrochloride.

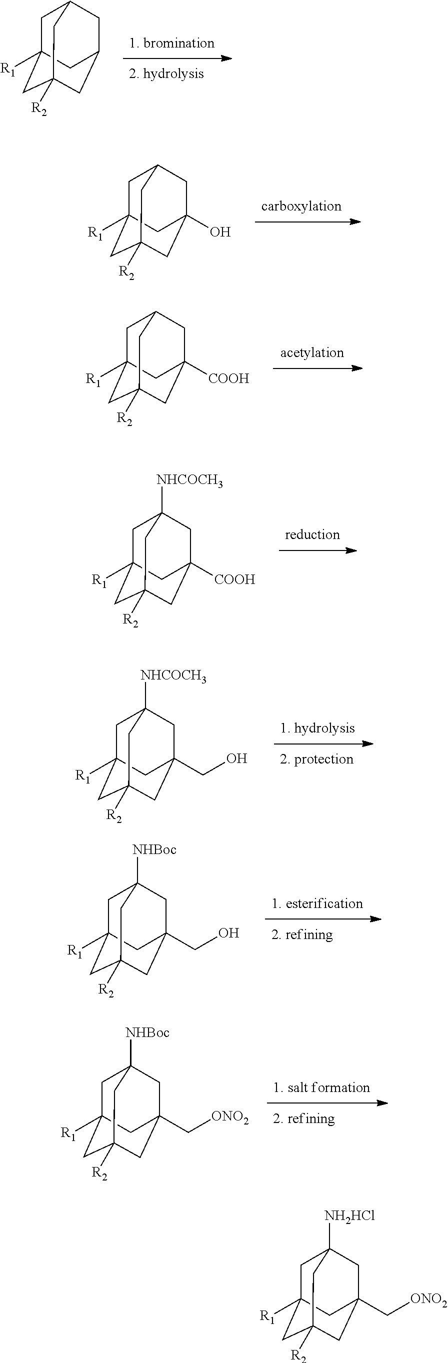

Wherein, R₁ and R₂ are each independently hydrogen, straight-chain or branched-chain alkyl, substituted or unsubstituted aryl or heteroaryl.

In the above process of manufacture, the raw material adamantane has the structure (A):

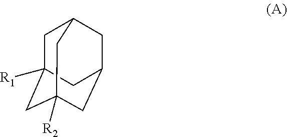

wherein R₁ and R₂ are each independently hydrogen, straight-chain or branched-chain alkyl, substituted or unsubstituted aryl or heteroaryl.

In various embodiments of the invention, the process of manufacture may include the following technical features.

In the process of manufacture, step (1) further includes following reactions:
(1a) Bromination of adamantane: reacting a substituted or unsubstituted adamantane with liquid bromine in reflux for 4-6 h to obtain bromoadamantane; and
(1b) Hydrolysis of bromoadamantane: adding sodium oxalate and water, the reaction system being refluxed at 75° C. via hydrolysis to obtain a substituted or unsubstituted adamantanol.

In the process of manufacture, step (2) further comprises: the product from step (1) being dissolved in formic acid, and slowly added dropwise to concentrated sulfuric acid with cooling to 0-10° C., and then the reaction being maintained at 0-10° C. for 3-6 h; the reaction solution being poured into ice water and stirred for 16-18 h; after suction filtration, the solid filter cake being washed with water, dissolved with 0.27× sodium hydroxide solution, and filtered with suction; the filtrate being acidified with 5-10% hydrochloric acid to pH=1-2 and filtered, the filter cake being washed with water until the filtrate being neutral, and dried at 40-50° C. to obtain adamantanoic acid.

In the process of manufacture, step (3) further comprises: cooling concentrated sulfuric acid to 0-10° C., and adding adamantanoic acid to be dissolved with stirring, adding slowly dropwise nitric acid, and then adding dropwise acetonitrile, reaction being maintained at 0-10° C. for 2-3 h; reaction solution being poured into ice water with stirring for 16-18 h, and filtered with suction, and filter cake being washed with water and dried at 40-50° C. to obtain amido adamantanic acid.

In the process of manufacture, step (4) further comprises: adding the product of step (3) to tetrahydrofuran, cooled to 0-10° C., and adding triethylamine in batches, then adding dropwise ethyl chloroformate, and reaction being run at room temperature for 4-6 h; filtering resulting reaction mixture, and washing filter cake with tetrahydrofuran, combing tetrahydrofuran phases and cooling to 0-10° C., adding sodium borohydride in batches, then adding 0.8-1× water dropwise, and reaction being run for 2-3 h; adding 5× water, filtering reaction mixture, filtrate being spin-dried, then extracted with ethyl acetate, and combined ethyl acetate phases being dried over sodium sulfate, filtered, and spin dried, and residue being added with 1.5-2.5× ethyl acetate, stirred well, the same amount of petroleum ether being added, and stirred for 12-16 h, filtered and filter cake dried at 40-50° C. to obtain an amido adamantanol.

In the process of manufacture, step (5) further comprises following reactions:
(5a) Hydrolysis: adding amido adamantanol and a strong base sequentially to polyethylene glycol with a 5-10 times volume, reacted via high temperature hydrolysis at 100-180° C. for 10-18 h, and reaction system being cooled, diluted with purified water and stirred evenly, and water layer being extracted with toluene, dried over sodium sulfate and filtered, and the filtrate being concentrated;
(5b) Condensation: dissolving the product obtained in reaction (5a) in dichloromethane, adding Boc anhydride, then running reaction via condensation at 20-30° C. for 1-5 h, and resulting mixture being concentrated to remove dichloromethane.

in the process of manufacture, wherein step (6) further comprises: adding n-Hexane to resulting residual oily liquid of step (5), reacted via crystallization for 2-3 h, and the resulting mixture being centrifuged, washed with n-hexane, and then dried at 40-50° C. for 16-18 h to obtain Boc-protected amantadinol.

In the process of manufacture, step (7) further comprises: mixing fuming nitric acid with acetic anhydride at a temperature of −10° C. to 10° C. to give a nitrating reagent; dissolving resulting solid product from step (6) in dichloromethane with temperature controlled at −10° C. to 10° C., adding dropwise the nitrating reagent to be reacted for 15 min to 6 h, then quencing reaction upon its completion with ice water and water layer being separated, and organic layer being washed respectively with 10 times volume (10 Vol) of a solution of saturated sodium bicarbonate and a solution of saturated sodium chloride, dried over sodium sulfate, filtered, and concentrated to remove dichloromethane to obtain a crude product of Boc-protected amantadine nitrate

In the process of manufacture, step (8) further comprises: dissolving the crude product of step (7) in alcohol, adding water for crystallization with mashing for 2-3 h, then the resulting mixture being filtered, and resulting solid being dried at 40-50° C. to obtain a refined product.

In the process of manufacture, step (9) further comprises: dissolving the product of step (8) in ethyl acetate with temperature controlled at 10-30° C., adding HCl/ethyl acetate, running reaction for 16-18 h, and then resulting mixture being filtered with suction, and dried to obtain an amantadine nitrate hydrochloride.

In the process of manufacture, step (10) further comprises: dissolving product of step (9) in ethanol, treating via filtration with filtrate being concentrated, and adding ethyl acetate for crystallization with beating, resulting mixture being dried to constant weight to obtain a refined amantadine nitrate hydrochloride.

In the process of manufacture, in reaction (1a), the molar ratio of reaction materials is raw material:bromine=1: 2-6, reaction temperature is 60-90° C., and reaction time is 4-6 h;

In the process of manufacture, in reaction (1b), the molar ratio of reaction materials is raw material:sodium oxalate=1:2.5-6.0, the amount of water used is 23-35×, and reaction time is 2-3 h.

In the process of manufacture, step (1), the post-treatment of step (1) comprises: adding 3-5× sodium sulfite solution to reaction solution, stirring at room temperature for 16-18 h and then filtered, and filter cake being washed with water and then dissolved with 3-8× organic solvent, dried over anhydrous sodium sulfate, and filtered, and filter cake being washed with 2-5× organic solvent, and spin-dried under reduced pressure to obtain adamantanol as a solid; wherein, the organic solvents are preferably methyl tert-butyl ether, ethyl acetate, dichloromethane and the like.

In the process of manufacture, step (2), the molar ratio of reaction feed is raw material:formic acid:sulfuric acid=1: 6-10:11-16, wherein the molar ratio of reaction feed is preferably raw material:formic acid:sulfuric acid=1: 6-8:11.5-13, and the reaction time is preferably 4-5 h.

In the process of manufacture, step (3), the molar ratio of reaction feed is raw material:nitric acid:sulfuric acid:acetonitrile=1:1.6-2.5:9-15:1.1-1.5.

In the process of manufacture, step (4), the molar ratio of reaction feed is raw material:triethylamine:ethyl chloroformate:sodium borohydride=1:1.2-1.5:1.2-1.5:1.5-2.5.

In the process of manufacture, step (5), the strong base is lithium hydroxide, sodium hydroxide, or potassium hydroxide, and the molar ratio of reaction feed is raw material:base=1: 5-10.

In the process of manufacture, step (5), the reaction temperature is 100-180° C., and the reaction time is preferably 15-16 h.

In the process of manufacture, step (5), the amount of dichloromethane is 5-10 times the volume (5-10 Vol) of the raw material, and the molar ratio of reaction feed is raw material:Boc anhydride=1:1.05-1.5, and the reaction time is 1-5 h.

In the process of manufacture, step (6), the amount of n-hexane used for crystallization of the product after condensation is 5-9 times in volume (5-9 Vol) of the raw material.

In the process of manufacture, step (7), the molar ratio of reaction fee is raw material:acetic anhydride:fuming nitric acid=1:1.08-1.8:1.68-2.8.

In the process of manufacture, step (7), the nitrating reagent is prepared at −10-10° C., and then stirred for 0.5-1 h.

In the process of manufacture, step (7), the reaction temperature is 0-10° C., and the reaction time is 15 min-6 h, preferably 30 min-3 h.

In the process of manufacture, step (8), the alcohol can be methanol or ethanol, and the weight ratio of reaction feed is raw material:alcohol 1:water=1: 2-5:3-8.

In the process of manufacture, step (8), after the oil obtained from step (7) is completely dissolved in ethanol, a portion of water is added with stirring until precipitation of a white solid, stirred for 0.5-1 h, and then the remaining water is added with beating for 1-2 h.

In the process of manufacture, step (8), the crystallization temperature is controlled at 20-30° C.

In the process of manufacture, step (9), the concentration of HCl in HCl/ethyl acetate is 2.0-4.37 M.

In the process of manufacture, step (9), the molar ratio of raw materials:HCl=1: 5-10, the reaction temperature is 20-30° C., and the reaction time is 16-18 h.

In the process of manufacture, step (9), the refined compound in step (8) is first dissolved in 3-6 times the weight of ethyl acetate, and then HCl/ethyl acetate is added for the reaction.

In the process of manufacture, step (10), the crude material is dissolved in alcohol, then concentrated under reduced pressure to 0.5-1 times the remaining ethanol in the system, and ethyl acetate is added with beating for 2-3 h.

In the process of manufacture, step (10), the weight ratio of the reaction feed is raw material:ethanol:ethyl acetate=1:2.5-3: 15-25.

In the process of manufacture, step (10), the temperature of ethanol for concentration is controlled at 40-50° C., and the drying temperature is controlled at 45-50° C.

As compared with the prior technology, the process of the present invention has significant advantages, including:

1) Through the optimization of the process, the amount of chemical reagents such as bromine, strong acids, strong bases used in the process was reduced, and the production cost was reduced. The waste liquids generated in the post-treatment and the production processes were reduced, and thus the process becomes more environmentally friendly;

2) In the step of hydrolysis, polyethylene glycol 400 is selected as the reaction solvent, which lowered the reaction temperature, reduced energy consumption, and made the process more environmentally friendly;

3) In the nitrification reaction, by controlling the amount of fuming nitric acid, the reaction speed and reaction time were adjusted, which reduced the formation of by-products, increased the output of the main product, and made the process more suitable for industrial production; furthermore, through the refining process, the post-treatment process was simplified, and the amount of other organic reagents was reduced;

4) In the salt-forming reaction, the amount of HCl is reduced, the refining process is simplified, and the impurities after washing are reduced, and thus the process is more environmentally friendly and suitable for industrial production.

Generally, the process of the present invention reduced the amount of reaction reagents in each step and reduced production costs. Also, the processes such as crystallization and filtration were used in the post-treatment, and the column chromatography separation and purification in the laboratory process were abandoned, and the purification process in post-treatment was simplified. More importantly, the process of the present invention suspended the nitrification reaction speed through optimization in the amount of nitrating reagents and appropriate extension of reaction time, and thus the conversion of main products to by-products is significantly reduced within 30 min-6 h, which can meet the requirements of time control in industrial production, and increase yield, reduce by-products, and make the overall process more green and environmentally friendly, with great industrial and socio-economic values. Therefore, the process for manufacture of the present invention has the advantages of low production costs, improved safe and reliability, and high reaction yields, and is very suitable for industrial production.

The following examples are used to further explain the present invention. It should be pointed out that for those of ordinary skills in the art, without departing from the concept of the present invention, certain improvements and modifications can be made, and these improvements and modifications should be regarded as within the scope of the present invention.

### DETAILED DESCRIPTION OF THE EMBODIMENTS

In the following embodiments, the process for manufacture of compound MN-08 will describe in more detail as examples of the technologies of the invention. Obviously, they are shown as only a part but not all of the embodiments of the present invention. Based on the embodiments shown here, all other embodiments obtained by those of ordinary skills in the art without creative work shall fall within the scope of the present invention.

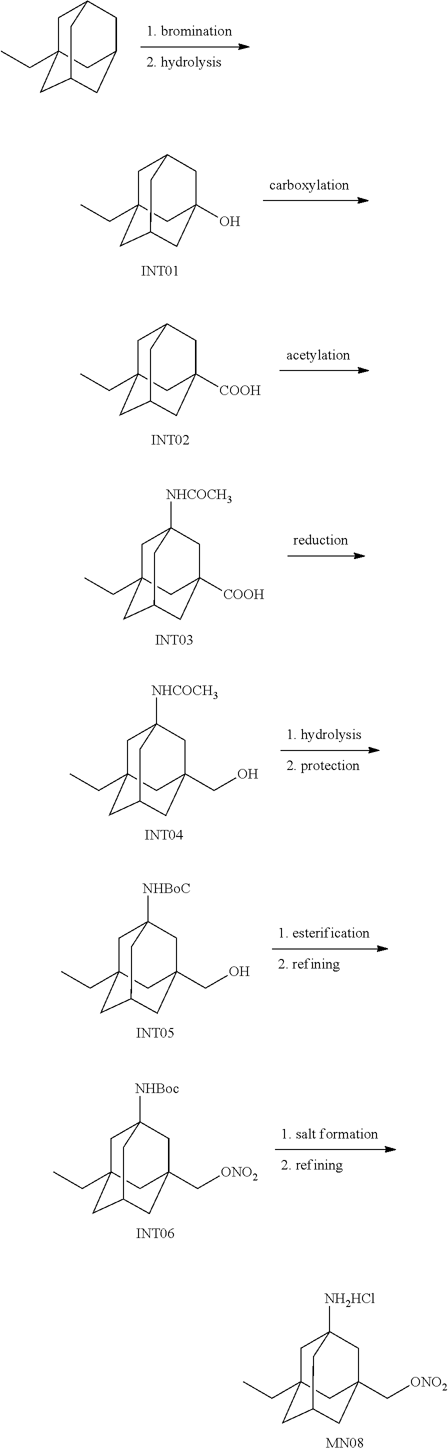

### Example 1. Preparation of INT01

In a 2 L round bottom flask was placed 100 g (0.61 mol) of ethyladamantane, 495 g (5×, 2.9 mol) of Br₂ was added, and the reaction was refluxed at 60° C. for 4 hours. The reaction solution was cooled to room temperature, and 500 g (5×, 3.6 mol) of (COONa)₂ and 800 mL (8×) of water were added, then heated to 75° C. and refluxed for 2 h. The reaction was monitored via TLC. After the reaction was completed, the reaction solution was cooled to room temperature, and then was added into a solution of saturated sodium sulfite and stirred for 16 h. The resulting mixture was filtered with suction, and the filter cake was washed with water and dissolved in 400 g (4×) of ethyl acetate, which was dried over 100 g (1×) of anhydrous sodium sulfate. After filtration, the filter residue was washed with 200 g of ethyl acetate, and the combined ethyl acetate layers were spin-dried under reduced pressure to give 102 g of INT01 as a solid with a yield of 92%.

### Example 2. Preparation of INT01

In a 2 L round bottom flask was placed 100 g (0.61 mol) of ethyladamantane, 307 g (3×, 1.8 mol) of Br₂ was added, and the reaction was refluxed at 75° C. for 4 hours. The reaction solution was cooled to room temperature, and 400 g (4×, 2.4 mol) of (COONa)₂ and 640 mL (6.4×) of water were added, then heated to 75° C. and refluxed for 2 h. The reaction was monitored via TLC. After the reaction was completed, the reaction solution was cooled to room temperature, and then was added into a solution of saturated sodium sulfite and stirred for 16 h. The resulting mixture was filtered with suction, and the filter cake was washed with water and dissolved in 400 g (4×) of ethyl acetate, which was dried over 100 g (1×) of anhydrous sodium sulfate. After filtration, the filter residue was washed with 200 g of ethyl acetate, and the combined ethyl acetate layers were spin-dried under reduced pressure to give 98 g of INT01 as a solid with a yield of 89%.

### Example 3. Preparation of INT01

In a 20 L reaction kettle was placed 1500 g (9.1 mol) of ethyladamantane, 4665 g (3.1×, 29.3 mol) of Br₂ was added, and the reaction was refluxed at 60° C. for 4 hours. The reaction solution was cooled to room temperature, and 3000 g (2×, 22.3 mol) of (COONa)₂ and 3000 mL (2×) of water were added, then heated to 75° C. and refluxed for 2 h. The reaction was monitored via TLC. After the reaction was completed, the reaction solution was cooled to room temperature, and then was added into a solution of saturated sodium sulfite (4500 g, 3×) and stirred for 16 h. The resulting mixture was filtered with suction, and the filter cake was washed with water and dissolved in 6000 g (4×) of ethyl acetate, which was dried over 1000 g (0.66×) of anhydrous sodium sulfate. After filtration, the filter residue was washed with 2000 g of ethyl acetate, and the combined ethyl acetate layers were spin-dried under reduced pressure to give 1500 g of INT01 as a solid with a yield of 91%.

### Example 4. Preparation of INT01

In a 500 L reaction kettle was placed 44 kg (269 mol) of ethyladamantane, 141 kg (3.2×, 542.7 mol) of Br₂ was added, and the reaction was refluxed at 60° C. for 4 hours. The reaction solution was cooled to room temperature, and 90 kg (2.04×, 3.6 mol) of (COONa)₂ and 112 L (2.55×) of water were added, then heated to 75° C. and refluxed for 2 h. The reaction was monitored via TLC. After the reaction was completed, the reaction solution was cooled to room temperature, and then was added into a solution of saturated sodium sulfite solution (135.0 kg, 3.05×) and stirred for 16 h. The resulting mixture was filtered with suction, and the filter cake was washed with water and dissolved in 343 kg (7.8×) of methyl tert-butyl ether, which was dried over 22 kg (0.5×) of anhydrous sodium sulfate. After filtration, the filter residue was washed with 88 kg (2×) of ethyl acetate and the combined organic layers were spin-dried under reduced pressure to obtain 45 kg of INT01 as a solid with a yield of 93%.

### Example 5. Preparation of INT02

In a 1 L round bottom flask was placed 400 mL (7.4×, 13 eq) of concentrated sulfuric acid, which was cooled to 0-10° C. in an ice water bath. In 140 mL (1.7×, 6.6 eq) of formic acid was dissolved with 100 g (0.56 mol) of INT01, and the mixture was then slowly dripped into the sulfuric acid, and after the addition, the reaction was run at 0-10° C. for 4 h. The reaction was monitored via TLC. After the reaction was completed, the reaction solution was slowly added dropwise to 2 L (20×) of ice water and stirred for 16 h. The resulting mixture was filtered with suction, and the filter cake was washed ice water until pH being neutral. The filter cake was resuspended in a solution of 27 g (0.27×, 1.2 eq) of sodium hydroxide in 600 mL (6×) of water, and then filtered, and the filter residue was washed with water. The aqueous phases were combined, added with water to be diluted to 1 L, and acidified with 10% HCl to pH=1-2, and then stirred for 4-5 h. After suction filtration, the filter cake was washed with water until pH being neutral, and dried at 40-50° C. to obtain 102 g of INT02 as a white solid with a yield of 90%.

### Example 6. Preparation of INT02

In a 5 L three-necked flask was placed 4360 g (8.7×, 16 eq) of concentrated sulfuric acid, which was cooled to 0-10° C. in an ice water bath. In 1000 g (2×, 7.8 eq) formic acid was dissolved with 500 g (2.78 mol) of INT01, and the mixture was then slowly dripped into the sulfuric acid, and after the addition, the reaction was run at 0-10° C. for 6 h. The reaction was monitored via TLC. After the reaction was completed, the reaction solution was slowly added dropwise to 20 L (40×) of ice water and stirred for 16 h. The resulting mixture was filtered with suction, and the filter cake was washed ice water until pH being neutral. The filter cake was resuspended in a solution of 133 g (0.27×, 1.2 eq) of sodium hydroxide in 5000 L (10×) of water, and then filtered, and the filter residue was washed with water. The aqueous phases were combined, added with water, and acidified with 10% HCl to pH=1-2, and then stirred for 4-5 h. After suction filtration, the filter cake was washed with water until pH being neutral, and dried at 40-50° C. to obtain 485 g of INT02 as a white solid with a yield of 84%.

### Example 7. Preparation of INT02

In a 20 L reaction kettle was placed 6400 g (6.4×, 11.5 eq) of concentrated sulfuric acid, which was cooled to 0-10° C. in an ice water bath. In 1700 g (1.7×, 6.6 eq) of formic acid was dissolved with 1000 g (5.6 mol) of INT01, and the mixture was then slowly dripped into the sulfuric acid, and after the addition, the reaction was run at 0-10° C. for 4 h. The reaction was monitored via TLC. After the reaction was completed, the reaction solution was slowly added dropwise to 20 L (20×) of ice water and stirred for 16 h. The resulting mixture was filtered with suction, and the filter cake was washed ice water until pH being neutral. The filter cake was resuspended in a solution of 270 g (0.27×, 1.2 eq) of sodium hydroxide in 10 L (10×) of water, and then filtered, and the filter residue was washed with water. The aqueous phases were combined, added with water, and acidified with 10% HCl to pH=1-2, and then stirred for 4-5 h. After suction filtration, the filter cake was washed with water until pH being neutral, and dried at 40-50° C. to obtain 980 g of INT02 as a white solid with a yield of 85%.

### Example 8. Preparation of INT02

In a 500 L reaction kettle was placed 288 kg (6.4×, 11.5 eq) of concentrated sulfuric acid, which was cooled to 0-10° C. in an ice water bath. In 77 kg (1.7×, 6.6 eq) of formic acid was dissolved with 45 kg (250 mol) of INT01, and the mixture was then slowly dripped into the sulfuric acid, and after the addition, the reaction was run at 0-10° C. for 4 h. The reaction was monitored via TLC. After the reaction was completed, the reaction solution was slowly added dropwise to 900 L (20×) of ice water and stirred for 16 h. The resulting mixture was filtered with suction, and the filter cake was washed ice water until pH being neutral. The filter cake was resuspended in a solution of 12.15 kg (0.27×, 1.2 eq) of sodium hydroxide in 250 kg (5.56×) of water, and then filtered, and the filter residue was washed with water. Concentrated hydrochloric acid was diluted from 35.1 kg to 185 kg, and was slowly added dropwise to the filtrate, and stirred for 4-5 h. After suction filtration, the filter cake was washed with water until pH being neutral, and dried at 40-50° C. to obtain 45.7 kg of INT02 as a white solid with a yield of 88%.

### Example 9. Preparation of INT03

In a 1 L three-necked flask in an ice water bath with cooling, was placed 75 mL of nitric acid (1.1×, 2.2 eq), which was then cooled to 0-10° C. in an ice water bath. To the nitric acid was added 100 g (0.48 mol) of INT02, and stirred well. To the reaction solution was slowly added dropwise 350 mL (6.4×, 13 eq) of concentrated sulfuric acid, and after the addition, the reaction was run with an ice-water bath for 1 h. Then, to the reaction solution was slowly added dropwise 31 mL (0.24×, 1.2 eq) of acetonitrile, and the reaction was continued to run with an ice water bath for 1 h. The reaction was monitored via TLC. After the reaction was complete, the reaction solution was poured into 2000 mL of ice-water mixture and stirred overnight. After suction filtration, the filter cake was washed with water until the filtrate being neutral and dried to obtain 107 g of INT03 as a white solid with a yield of 83.9%.

### Example 10 Preparation of INT03

In a 5 L three-necked flask in an ice water bath with cooling, was placed 137.5 mL of nitric acid (0.78×, 1.7 eq), which was then cooled to 0-10° C. in an ice water bath. To the nitric acid was added 250 g (1.2 mol) of INT02, and stirred well. To the reaction solution was slowly added dropwise 1700 mL (12×, 27 eq) of concentrated sulfuric acid, and after the addition, the reaction was run at 0-10° C. for 1 h. Then, to the reaction solution was slowly added dropwise 125 mL (0.4×, 2 eq) of acetonitrile, and the reaction was continued to run at 0-10° C. for 1 h. The reaction was monitored via TLC. After the reaction was complete, the reaction solution was poured into 5000 mL of ice-water mixture and stirred overnight. After suction filtration, the filter cake was washed with water until the filtrate being neutral and dried to obtain 270 g of INT03 as a white solid with a yield of 84.7%.

### Example 11. Preparation of INT03

To a 20 L reaction kettle, turned on with cooling, was placed 500 mL (0.7×, 1.6 eq) of nitric acid, which was then cooled to 0-10° C. in an ice water bath. To the nitric acid was added 1000 g (4.8 mol) of INT02, and stirred well. To the reaction solution was slowly added dropwise 3500 mL (6.4×, 13 eq) of concentrated sulfuric acid, and after the addition, the reaction was run at 0-10° C. for 1 h. Then, to the reaction solution was slowly added dropwise 310 mL (0.24×, 1.2 eq) of acetonitrile, and the reaction was continued to run at 0-10° C. for 1 h. The reaction was monitored via TLC. After the reaction was complete, the reaction solution was poured into 2000 mL of ice-water mixture and stirred overnight. After suction filtration, the filter cake was washed with water until the filtrate being neutral and dried to obtain 1050 g of INT03 as a white solid with a yield of 82.4%.

### Example 12. Preparation of INT04

To 356 g (3.56×) of tetrahydrofuran was added 100 g (0.38 mmol) of INT03 with cooling in an ice water bath, and 76 g (0.76×, 0.76 mol, 2 eq) of triethylamine was added in batches. After the solid was completely dissolved, to the solution was added dropwise 82 g of ethyl chloroformate (0.82×, 0.76 mol, 2 eq). After the addition, the ice bath was removed, and the reaction was run at room temperature for 4 h. The reaction was monitored via TLC. The reaction solution was suction filtered, and the solid filter cake was washed twice with tetrahydrofuran (1×) and then the tetrahydrofuran collections were combined. To the reaction solution was added 43 g (0.43×, 1.14 mol, 3 eq) of NaBH₄ in batches, and after the addition, 100 mL of water was slowly added and the reaction was run for 1 h. The reaction was quenched by slowly adding 300 mL of water. After the solvent was evaporated under reduced pressure, the aqueous layer was extracted with ethyl acetate (4×100 mL). The extract was washed twice with 100 mL of saturated NaCl solution and dried with anhydrous Na₂SO₄. The resulting material was filtered and evaporated to remove the solvent under reduced pressure to obtain a crude oil. The oil was dissolved in 200 mL of ethyl acetate, and then 200 mL of petroleum ether was added, and the mixture was stirred for 16 h. After suction filtration, the filter cake was washed with 100 mL of a mixed solution of petroleum ether:ethyl acetate=1:1, and then dried at 40-50° C. to obtain 65 g of INT04 with a yield of 68.6%.

### Example 13, Preparation of INT04

To 890 g (3.56×) of tetrahydrofuran was added 250 g (0.94 mmol) of INT03 with cooling in an ice water bath, and 142 g (0.57×, 1.41 mol, 1.5 eq) of triethylamine was added in batches. After the solid was completely dissolved, to the solution was added dropwise 152 g (0.61×, 1.41 mol, 1.5 eq) of ethyl chloroformate. After the addition, the ice bath was removed, and the reaction was run at room temperature for 4 h. The reaction was monitored via TLC. The reaction solution was suction filtered, and the solid filter cake was washed twice with tetrahydrofuran (1×) and then the tetrahydrofuran collections were combined. To the reaction solution was added 71 g (0.28×, 1.88 mol, 2 eq) of NaBH₄ in batches, and after the addition, 200 mL of water was slowly added dropwise and the reaction was run for 1 h. The reaction was quenched by slowly adding 500 mL of water. After the solvent was evaporated under reduced pressure, the aqueous layer was extracted with ethyl acetate (4×250 mL). The extract was washed twice with 250 mL of saturated NaCl solution and dried with anhydrous Na₂SO₄. The resulting material was filtered and evaporated to remove the solvent under reduced pressure to obtain a crude oil. The oil was dissolved in 500 mL of ethyl acetate, and then 500 mL of petroleum ether was added, and the mixture was stirred for 16 h. After suction filtration, the filter cake was washed with 250 mL of a mixture solution of petroleum ether:ethyl acetate=1:1, and then dried at 40-50° C. to obtain 165 g of INT04 with a yield of 70%.

### Example 14, Preparation of INT04

To 3560 g (3.56×) of tetrahydrofuran was added 1000 g (3.8 mmol) of INT03 with cooling in an ice water bath, and 460 g (0.46×, 4.65 mol, 1.2 eq) of triethylamine was added in batches. to the solution in batches. After the solid was completely dissolved, to the solution was added dropwise 502 g (0.5×, 4.65 mol, 1.2 eq) of ethyl chloroformate. After the addition, the ice bath was removed, and the reaction was run at room temperature for 4 h. The reaction was monitored via TLC. The reaction solution was suction filtered, and the solid filter cake was washed twice with tetrahydrofuran (1×) and then the tetrahydrofuran collections were combined. To the reaction solution was added 230 g (0.23×, 6.08 mol, 1.6 eq) of NaBH₄ in batches, and after the addition, 800 mL of water was slowly added dropwise and the reaction was run for 1 h. The reaction was quenched by slowly adding 3000 mL of water. After the solvent was evaporated under reduced pressure, the aqueous layer was extracted with ethyl acetate (4×1000 mL). The extract was washed twice with 1000 mL of saturated NaCl solution and dried with anhydrous Na₂SO₄. The resulting material was filtered and evaporated to remove the solvent under reduced pressure to obtain a crude oil. The oil was dissolved in 2000 mL of ethyl acetate, and then 2000 mL of petroleum ether was added, and the mixture was stirred for 16 h. After suction filtration, the filter cake was washed with 1000 mL of a mixture solution of petroleum ether:ethyl acetate=1:1, and then dried at 40-50° C. to obtain 680 g of INT04 with a yield of 71.8%.

### Example 15. Preparation of INT05

(1) In a 5 L round bottom flask were placed 250 g (1 mol) of INT04 and 320 g (8 mol, 8 eq) of solid sodium hydroxide, and 1250 mL (5 Vol) of PEG-400 was added and refluxed at 120-130° C. for 16 h. The reaction was monitored via HPLC until INT04<0.5%, and then cooled to 70-80° C. To the reaction was added 2.5 L of water (10 Vol), and well stirred. The resulting material was extracted once with 2.5 L (10 Vol) of toluene, and then extracted twice with toluene (1250 mL, 5 Vol). The toluene collections were combined, and washed with saturated sodium chloride solution (2.5 L, 10 Vol), and dried with anhydrous sodium sulfate. After filtration, the filter residue was washed with toluene (250 mL, 1 Vol), and the solvent was evaporated under reduced pressure to obtain 223 g of gray crude solid.

(2) In 1.3 L of dichloromethane (6 Vol) was dissolved 223 g of the crude solid, and 229 g (1.05 mol, 1.05 eq) of Boc anhydride was added, and the reaction was run at 20-30° C. with stirring for 2 h, with HPLC monitoring until INT04<0.5%. After the reaction, the dichloromethane was evaporated under reduced pressure, and to the residue was added 1.6 L of n-hexane for crystallization for 3 h. After filtration, the filter cake was washed twice with 250 mL of n-hexane, and dried at 40-50° C. to constant weight to give 214 g of INT05 as a white solid with a total yield of 69.3%.

### Example 16. Preparation of INT05

(1) In a 5 L round bottom flask were placed 150 g (0.6 mol) of INT04 and 269 g (4.8 mol, 8 eq) of solid sodium hydroxide, and 750 mL (5 Vol) of PEG-400 was added and refluxed at 120-130° C. for 15 h. The reaction was monitored via HPLC until INT04<0.5%, and then cooled to 70-80^{c}ob to the reaction was added 1.5 L of water (10 Vol), and well stirred. The resulting material was extracted once with 1.5 L (10 Vole) of toluene, and then extracted twice with toluene (750 mL, 5 Vol). The toluene collections were combined, and washed with saturated sodium chloride solution (2.5 L, 10 Vol), and dried with anhydrous sodium sulfate. After filtration, the filter residue was washed with toluene (250 mL, 1 Vol), and the solvent was evaporated under reduced pressure to obtain 108 g of gray crude solid.

(2) In 640 mL of dichloromethane (8 Vol) was dissolved 81 g of the crude solid, and 119 g (0.57 mol, 1.5 eq) of Boo anhydride was added, and the reaction was run at 20-30° C. with stirring for 4 h, with HPLC monitoring until INT04<0.5%. After the reaction, the dichloromethane was evaporated under reduced pressure, and to the residue was added 400 mL of n-hexane for crystallization for 4 h. After filtration, the filter cake was washed twice with 80 mL of n-hexane, and dried at 40-50° C. to constant weight to give 80 g of INT05 as a white solid with a total yield of 66.8%.

### Example 17, Preparation of INT05

(1) In a 5 L round bottom flask were placed 150 g (0.6 mol) of INT04 and 144 g (3.6 mol, 6 eq) of solid potassium hydroxide, add 750 mL (5 Vol) of PEG-400 was added and refluxed at 120-130° C. for 16 h. The reaction was monitored via HPLC until INT04<0.5%, and then cooled to 70-80° C. To the reaction was added 1.5 L of water (10 Vol), and well stirred. The resulting material was extracted once with toluene (1.5 L, 10 Vol), and then extracted twice with toluene (750 mL, 5 Vol). The toluene collections were combined, and washed with saturated sodium chloride solution (2.5 L, 10 Vol), and dried with anhydrous sodium sulfate. After filtration, the filter residue was washed with toluene (250 mL, 1 Vol), and the solvent was evaporated under reduced pressure to obtain 103 g of gray crude solid.

(2) In 820 mL of dichloromethane (10 Vol) was dissolved 82 g of crude solid, and 86 g (0.42 mol, 1.05 eq) of Boc anhydride was added, and the reaction was run at 20-30° C. with stirring for 2 h, with HPLC monitoring until INT04<0.5%. After the reaction, the dichloromethane was evaporated under reduced pressure, and to the residue was added 550 mL of n-hexane for crystallization for 3 h. After filtration, the filter cake was washed twice with 80 mL of n-hexane, and dried at 40-50° C. to constant weight to give 75.5 g of INT05 as a white solid with a total yield of 62.3%.

### Example 18. Preparation of INT05

(1) In a 100 L reactor were placed (15.9 mol) of INT04 4 kg and 5.06 kg (126.5 mol, 8 eq) of solid sodium hydroxide, and 22.3 kg (5 Vol) of PEG-400 was added and refluxed at 120-130° C. for 16 h. The reaction was monitored via HPLC until INT04<2%, and then cooled to 70-80° C. To the reaction was added 40 L of water (10 Vol) to the reaction, and was extracted once with toluene (40 L, 10 Vol), and then extracted twice with toluene (20 L, 5 Vol). The toluene collections were combined, and washed with saturated sodium chloride solution (40 L, 10 Vol), and dried with anhydrous sodium sulfate. After filtration, the filter residue was washed with toluene (4 L, 1 Vol), and the solvent was evaporated under reduced pressure to obtain a gray crude solid.

(2) In 40 L of dichloromethane (10 Vol) was dissolved the crude solid, and 3.56 kg (16.3 mol, 1.05 eq) of Boc anhydride was added, and the reaction was run at 20-30° C. with stirring for 2 h, with HPLC monitoring until INT04<0.5%. After the reaction, the dichloromethane was evaporated under reduced pressure, and to the residue was added 15.1 kg of n-hexane for crystallization for 5 h. After filtration, the filter cake was washed twice with 2.1 kg n-hexane, and dried at 40-50° C. to constant weight to give 3.5 kg of INT05 as a white solid with a total yield of 71.1%.

### Example 19, Preparation of INT06

To 3.8 mL (40.8 mmol, 1.8 eq) of acetic anhydride, which was cooled to 0-10° C., was slowly added dropwise 2.8 mL (63.5 mmol, 2.8 eq) of fuming nitric acid, and then the resulting mixture was stirred for 30 min. A solid of INT05 (7 g, 22.7 mmol) was dissolved in 70 mL of dichloromethane with cooling to 0-10° C., and the prepared nitrating reagent was added dropwise. The reaction was kept at 0-10° C. and run for 6 h. Samples were taken at 0, 0.5, 1.5, 3, and 6 hours after the dropwise addition, and the reaction was monitored via HPLC to check at different time points the conditions of the raw materials, products and impurities, the results of which are shown in the table below. The raw materials were completed consumed after the dropwise addition, at the same time the by-products began to form, and the by-products reached 15% at 0.5 h of the reaction, INT06 dropped to 33% at 3 h of the reaction, and INT06 dropped to about 18% at 6 h of the reaction.

| Time | INT05 | INT06 | Impurities |
|---|---|---|---|
| 0 h | N.D. | 75.30% | 1.35% |
| 0.5 h | N.D. | 63.13% | 14.48% |
| 1.5 h | N.D. | 47.82% | 32.63% |
| 3 h | N.D. | 32.63% | 50.36% |
| 6 h | N.D. | 17.91% | 63.77% |

### Example 20. Preparation of INT06

To 3.42 mL (32.6 mmol, 1.62 eq) of acetic anhydride, which was cooled to 0-10° C., was slowly added dropwise 2.52 mL (57.5 mmol, 2.52 eq) of fuming nitric acid, and then the resulting mixture was stirred for 30 min. A solid of INT05 (7 g, 22.7 mmol) was dissolved in 70 mL of dichloromethane with cooling to 0-10° C., and the prepared nitrating reagent was added dropwise. The reaction was kept at 0-10° C. and run for 6 h. Samples were taken at 0, 0.5, 1.5, 3 and 6 hours after the dropwise addition, and the reaction was monitored via HPLC to check at different time points the conditions of the raw materials, products and impurities, the results of which are shown in the table below. The raw materials were completed consumed after the addition, at the same time the by-products began to form, and the by-products reached 6% at 0.5 h of the reaction, INT06 dropped to 56% at 3 h of the reaction, and INT06 dropped to about 43% at 6 h of the reaction

| Time | INT05 | INT06 | Impurities |
|---|---|---|---|
| 0 h | N.D. | 77.22% | 0.89% |
| 0.5 h | N.D. | 73.17% | 6.08% |
| 1.5 h | N.D. | 65.22% | 14.40% |
| 3 h | N.D. | 55.82% | 23.65% |
| 6 h | N.D. | 43.15% | 36.98% |

### Example 21. Preparation of INT06

To 3.04 mL (36.7 mmol, 1.44 eq) of acetic anhydride, which was cooled to 0-10° C., was slowly added dropwise 2.24 mL (50.8 mmol, 2.24 eq) of fuming nitric acid, and then the resulting mixture was stirred for 30 min. A solid of INT05 (7 g, 22.7 mmol) was dissolved in 70 mL of dichloromethane with cooling to 0-10° C., and the prepared nitrating reagent was added dropwise. The reaction was kept at 0-10° C. and run for 6 h. Samples were taken at 0, 0.5, 1.5, 3, and 6 hours after the dropwise addition, and the reaction was monitored via HPLC to check at different time points the conditions of the raw materials, products and impurities, the results of which are shown in the table below. The raw materials were completed consumed 0.5 h after the addition, and at the same time the by-products reached 3.5%, and the by-product was about 12% at 3 h of the reaction.

| Time | INT05 | INT06 | Impurities |
|---|---|---|---|
| 0 h | 0.68% | 76.55% | 0.32% |
| 0.5 h | N.D. | 75.24% | 3.50% |
| 1.5 h | N.D. | 73.88% | 6.30% |
| 3 h | N.D. | 67.42% | 11.73% |
| 6 h | N.D. | 58.22% | 20.77% |

### Example 22. Preparation of INT06

To 2.66 mL (28.6 mmol, 1.26 eq) of acetic anhydride, which was cooled to 0-10° C., was slowly added dropwise 1.96 mL (44.5 mmol, 1.96 eq) of fuming nitric acid, and then the resulting mixture was stirred for 30 min. A solid of INT05 (7 g, 22.7 mmol) was dissolved in 70 mL of dichloromethane with cooling to 0-10° C., and the prepared nitrating reagent was added dropwise. The reaction was kept at 0-10° C. and run for 6 h. Samples were taken at 0, 0.5, 1.5, 3, and 6 hours after the dropwise addition, and the reaction was monitored via HPLC to check at different time points the conditions of the raw materials, products and impurities, the results of which are shown in the table below. The raw material remained 0.37% at 0.5 h after the dropwise addition, which met the requirements of central control. The raw material reacted completely within 3 h of the reaction, and the by-product was 1.84%.

| Time | INT05 | INT06 | Impurities |
|---|---|---|---|
| 0 h | 3.42% | 71.20% | N.D. |
| 0.5 h | 0.37% | 77.13% | 0.94% |
| 1.5 h | 0.23% | 77.55% | 1.37% |
| 3 h | N.D. | 77.61% | 1.84% |
| 6 h | N.D. | 76.33% | 2.79% |

### Example 23. Preparation of INT06

To 2.28 mL (24.5 mmol, 1.08 eq) of acetic anhydride, which was cooled to 0-10° C., was slowly added dropwise 1.68 mL (38.1 mmol, 1.68 eq) of fuming nitric acid, and then the resulting mixture was stirred for 30 min. A solid of INT05 (7 g, 22.7 mmol) was dissolved in 70 mL of dichloromethane with cooling to 0-10° C., and the prepared nitrating reagent was added dropwise. The reaction was kept at 0-10° C. and run for 6 h. Samples were taken at 0, 0.5, 1.5, 3 and 6 hours after the dropwise addition, and the reaction was monitored via HPLC to check at different time points the conditions of the raw materials, products and impurities, the results of which are shown in the table below. The raw material remained 0.49% after the dropwise addition, and the by-product level was 0.78%, and after 6 h of the reaction the raw material completely consumed, and INT06 was about 79%.

| Time | INT05 | INT06 | Impurities |
|---|---|---|---|
| 0 h | 10.22% | 57.33% | N.D. |
| 0.5 h | 0.59% | 77.56% | 0.51% |
| 1.5 h | 0.72% | 78.06% | 0.69% |
| 3 h | 0.49% | 77.50% | 0.67% |
| 6 h | N.D. | 78.64% | 0.74% |

### Example 24. Preparation of INT06

(1) To 114 mL (1.2 mol, 1.8 eq) of acetic anhydride, which was cooled to 0-10° C., was slowly added dropwise 84 mL (1.9 mol, 2.8 eq) of fuming nitric acid, and then the resulting mixture was stirred for 30 min. A solid of 210 g (0.68 mol) of INT05 was dissolved in 2.1 L (10 Vol) of dichloromethane with cooling to 0-10° C., and the prepared nitrating reagent was added dropwise. The reaction was kept at 0-10° C. and run for 30 min. The reaction was monitored via HPLC until INT05<0.5%, and then 2.1 L of ice water (10 Vol) was added to quench the reaction. After stirring, the aqueous layer was separated, and the organic layer was washed with 2.1 L (10 Vol) of 1N sodium bicarbonate solution and 2.1 L (10 Vol) of saturated sodium chloride solution, and further dried with anhydrous sodium sulfate. After filtration, the filter residue was washed twice with 210 mL of dichloromethane. The dichloromethane phased were combined and concentrated to evaporate the solvent under reduced pressure at 30-35° C. to give a yellow oily liquid.

(2) To the yellow oily liquid was added 480 mL of ethanol, which was stirred until complete dissolution, and 30 mL of water was added. The reaction was controlled with the temperature at 20-30° C. and stirred until forming of crystals. The resulting suspension was added with remaining 420 mL of water, kept at 20-30° C., and stirred for 2 h. After suction filtration, the filter cake was sucked dry, and then dried in vacuum at 40-50° C. to constant weight to give 200 g of INT06 as a white solid with a yield of 83%.

### Example 25. Preparation of INT06

(1) To 14.6 mL (157 mmol, 1.62 eq) of acetic anhydride, which was cooled to 0-10° C., was slowly added dropwise 10.8 mL (244 mmol, 2.52 eq) of fuming nitric acid, and then the resulting mixture was stirred for 30 min. A solid of 30 g (97 mmol) of INT05 was dissolved in 300 mL (10 Vol) of dichloromethane with cooling to 0-10° C., and the prepared nitrating reagent was added dropwise. The reaction was kept at 0-10° C. and run for 30 min. The reaction was monitored via HPLC until INT05<0.5%, and then 300 mL of ice water (10 Vol) was added to quench the reaction. After stirring, the aqueous layer was separated, and the organic layer was washed with 300 mL (10 Vol) of 1N sodium bicarbonate solution and 300 mL (10 Vol) of saturated sodium chloride solution, and further dried with anhydrous sodium sulfate. After filtration, the filter residue was washed twice with 30 mL of dichloromethane. The dichloromethane phased were combined and concentrated to evaporate the solvent under reduced pressure at 30-35° C. to give a yellow oily liquid.

(2) To the yellow oily liquid was added 127 mL of ethanol, which was stirred until complete dissolution, and 30 mL of water was added. The reaction was controlled with the temperature at 20-30° C. and stirred until forming of crystals. The resulting suspension was added with remaining 60 mL of water, kept at 20-30° C., and stirred for 2 h. After suction filtration, the filter cake was sucked dry, and then dried in vacuum at 40-50° C. to constant weight to give 27.4 g of INT06 as a white solid with a yield of 80%.

### Example 26. Preparation of INT06

(1) To 13.0 mL (0.14 mol, 1.44 eq) of acetic anhydride, which was cooled to 0-10° C., was slowly added dropwise 9.6 mL (0.22 mol, 2.24 eq) of fuming nitric acid, and then the resulting mixture was stirred for 30 min. A solid of 30 g (97 mmol) of INT05 was dissolved in 300 mL (10 Vol) of dichloromethane with cooling to 0-10° C., and the prepared nitrating reagent was added dropwise. The reaction was kept at 0-10° C. and run for 30 min. The reaction was monitored via HPLC until INT05<0.5%, and then 300 mL of ice water (10 Vol) was added to quench the reaction. After stirring, the aqueous layer was separated, and the organic layer was washed with 300 mL (10 Vol) of 1N sodium bicarbonate solution and 300 mL (10 Vol) of saturated sodium chloride solution, and further dried with anhydrous sodium sulfate. After filtration, the filter residue was washed twice with 30 mL of dichloromethane. The dichloromethane phased were combined and concentrated to evaporate the solvent under reduced pressure at 30-35° C. to give a yellow oily liquid.

(2) To the yellow oily liquid was added 127 mL of ethanol, which was stirred until complete dissolution, and 30 mL of water was added. The reaction was controlled with the temperature at 20-30° C. and stirred until forming of crystals. The resulting suspension was added with remaining 60 mL of water, kept at 20-30° C., and stirred for 2 h. After suction filtration, the filter cake was sucked dry, and then dried in vacuum at 40-50° C. to constant weight to give 26.8 g of INT06 as a white solid with a yield of 78%.

### Example 27. Preparation of INT06

(1) To 15.2 mL (0.16 mol, 1.26 eq) of acetic anhydride, which was cooled to 0-10° C., was slowly added dropwise 11.2 mL (0.25 mol, 1.96 eq) of fuming nitric acid, and then the resulting mixture was stirred for 30 min. A solid of 40 g (0.13 mol) of INT05 was dissolved in 400 mL (10 Vol) of dichloromethane with cooling to 0-10° C., and the prepared nitrating reagent was added dropwise. The reaction was kept at 0-10° C. and run for 6 h. The reaction was monitored via HPLC until INT05<0.5%, and then 400 mL of ice water (10 Vol) was added to quench the reaction. After stirring, the aqueous layer was separated, and the organic layer was washed with 400 mL (10 Vol) of 1N sodium bicarbonate solution and 400 mL (10 Vol) of saturated sodium chloride solution, and further dried with anhydrous sodium sulfate. After filtration, the filter residue was washed twice with 40 mL of dichloromethane. The dichloromethane phased were combined and concentrated to evaporate the solvent under reduced pressure at 30-35° C. to give a yellow oily liquid.

(2) To the yellow oily liquid was added 157 mL of ethanol, which was stirred until complete dissolution, and 40 mL of water was added. The reaction was controlled with the temperature at 20-30° C. and stirred until forming of crystals. The resulting suspension was added with remaining 80 mL of water, kept at 20-30° C., and stirred for 2 h. After suction filtration, the filter cake was sucked dry, and then dried in vacuum at 40-50° C. to constant weight to give 40.7 g of INT06 as a white solid with a yield of 89%.

### Example 28. Preparation of INT06

(1) To 13.0 mL (0.14 mol, 1.08 eq) of acetic anhydride, which was cooled to 0-10° C., was slowly added dropwise 9.6 mL (0.22 mol, 1.68 eq) of fuming nitric acid, and then the resulting mixture was stirred for 30 min. A solid of 40 g (0.13 mol) of INT05 was dissolved in 400 mL (10 Vol) of dichloromethane with cooling to 0-10° C., and the prepared nitrating reagent was added dropwise. The reaction was kept at 0-10° C. and run for 6 h. The reaction was monitored via HPLC until INT05<0.5%, and then 400 mL of ice water (10 Vol) was added to quench the reaction. After stirring, the aqueous layer was separated, and the organic layer was washed with 400 mL (10 Vol) of 1N sodium bicarbonate solution and 400 mL (10 Vol) of saturated sodium chloride solution, and further dried with anhydrous sodium sulfate. After filtration, the filter residue was washed twice with 40 mL of dichloromethane. The dichloromethane phased were combined and concentrated to evaporate the solvent under reduced pressure at 30-35° C. to give a yellow oily liquid.

(2) To the yellow oily liquid was added 157 mL of ethanol, which was stirred until complete dissolution, and 40 mL of water was added. The reaction was controlled with the temperature at 20-30° C. and stirred until forming of crystals. The resulting suspension was added with remaining 80 mL of water, kept at 20-30° C., and stirred for 2 h. After suction filtration, the filter cake was sucked dry, and then dried in vacuum at 40-50° C. to constant weight to give 38.9 g of INT06 as a white solid with a yield of 85%.

### Example 29. Preparation of INT06

(1) To 1.45 kg (14.3 mol, 1.26 eq) of acetic anhydride, which was cooled to 0-10° C., was slowly added dropwise 1.47 kg (22.17 mol, 1.96 eq) of fuming nitric acid, and then the resulting mixture was stirred for 30 min. A solid of 3.5 kg (11.3 mol) of INT05 was dissolved in 35 L (10 Vol) of dichloromethane with cooling to 0-10° C., and the prepared nitrating reagent was added dropwise. The reaction was kept at 0-10° C. and run for 0.5 h. The reaction was monitored via HPLC until INT05<0.5%, and then 5 L of ice water (10 Vol) was added to quench the reaction. After stirring, the aqueous layer was separated, and the organic layer was washed with 35 L (10 Vol) of 1N sodium bicarbonate solution and 35 L (10 Vol) of saturated sodium chloride solution, and further dried with anhydrous sodium sulfate. After filtration, the filter residue was washed twice with 35 L of dichloromethane. The dichloromethane phased were combined and concentrated to evaporate the solvent under reduced pressure at 30-35° C. to give a yellow oily liquid.

(2) To the yellow oily liquid was added 13.65 kg of ethanol, which was stirred until complete dissolution, and 3.5 L of water was added. The reaction was controlled with the temperature at 20-30° C. and stirred until forming of crystals. The resulting suspension was added with remaining 7 L of water, kept at 20-30° C., and stirred for 2 h. After suction filtration, the filter cake was sucked dry, and then dried in vacuum at 40-50° C. to constant weight to give 3.8 kg of INT06 as a white solid with a yield of 94%.

### Example 30. Preparation of MN08

(1) HCl gas was introduced into ethyl acetate to form an HCl/ethyl acetate solution with a concentration of 4.37 M. In 1 L of ethyl acetate was dissolved 200 g (0.56 mol) of INT06, and 646 mL of HCl/ethyl acetate solution (HCl 2.8 mol, 5 eq) was added. The reaction was controlled with the temperature at 20-30° C. and run for 16 h. The reaction was monitored via HPLC, and terminated when INT06<0.5%. The resulting mixture was filtered with suction, and the filter cake was washed with 400 mL of ethyl acetate, and sucked to dry to give crude MN08.

(2) The crude MN08 was dissolved completely in 500 mL of ethanol and filtered with suction, and the filter cake was washed with 80 mL of ethanol. The combined ethanol was distilled under reduced pressure at 40-50° C. to a residue with 100 mL of ethanol remaining, the temperature was lowered to 20-30° C., and 4 L of ethyl acetate was added with stirring for 2 h. The resulting mixture was filtered with suction, and the filter cake was washed with 200 mL of ethyl acetate, and dried under reduced pressure at 40-50° C. to constant weight, to give 128 g of MN08 as a white solid with a yield of 78%.

### Example 31. Preparation of MN08

(1) HCl gas was introduced into ethyl acetate to form an HCl/ethyl acetate solution with a concentration of 3.7 M. In 465 mL of ethyl acetate was dissolved 93 g (0.26 mol) of INT06, and 700 mL of HCl/ethyl acetate solution (2.6 mol HCl, 10 eq) was added. The reaction was controlled with the temperature at 20-30° C. and run for 16 h. The reaction was monitored via HPLC, and terminated when INT06<0.5%. The resulting mixture was filtered with suction, and the filter cake was washed with 190 mL of ethyl acetate, and sucked to dry to give crude MN08.

(2) The crude MN08 was dissolved completely in 280 mL of ethanol and filtered with suction, and the filter cake was washed with 80 mL of ethanol. The combined ethanol was distilled under reduced pressure at 40-50° C. to a residue with 50 mL of ethanol remaining, the temperature was lowered to 20-30° C., and 1.4 L of ethyl acetate was added with stirring for 2 h. The resulting mixture was filtered with suction, and the filter cake was washed with 100 mL of ethyl acetate, and dried under reduced pressure at 40-50° C. to constant weight, to give 58 g of MN08 as a white solid with a yield of 76%.

### Example 32. Preparation of MN08

(1) HCl gas was introduced into ethyl acetate to form an HCl/ethyl acetate solution with a concentration of 3.7 M. In 590 mL of ethyl acetate was dissolved 118 g (0.33 mol) of INT06, and 446 mL of HCl/ethyl acetate solution (1.65 mol HCl, 5 eq) was added. The reaction was controlled with the temperature at 20-30° C. and run for 16 h. The reaction was monitored via HPLC, and terminated when INT06<0.5%. The resulting mixture was filtered with suction, and the filter cake was washed with 259 mL of ethyl acetate, and sucked to dry to give crude MN08.

(2) The crude MN08 was dissolved completely in 360 mL of ethanol and filtered with suction, and the filter cake was washed with 120 mL of ethanol. The combined ethanol was distilled under reduced pressure at 40-50° C. to a residue with 100 mL of ethanol remaining, the temperature was lowered to 20-30° C., and 2.4 L of ethyl acetate was added with stirring for 2 h. The resulting mixture was filtered with suction, and the filter cake was washed with 200 mL of ethyl acetate, and dried under reduced pressure at 40-50° C. to constant weight, to give 70 g of MN08 as a white solid with a yield of 83%.

### Example 33. Preparation of MN08

(1) HCl gas was introduced into ethyl acetate to form an HCl/ethyl acetate solution with a concentration of 4.37 M. In 22.5 L of ethyl acetate was dissolved 4.5 kg (12.7 mol) of INT06, and 14.5 L of HCl/ethyl acetate solution (63.6 mol HCl, 5 eq) was added. The reaction was controlled with the temperature at 20-30° C. and run for 16 h. The reaction was monitored via HPLC, and terminated when INT06<0.5%. The resulting mixture was filtered with suction, and the filter cake was washed with 9 L of ethyl acetate, and sucked to dry to give crude MN08.

(2) The crude MN08 was dissolved completely in 12 L of ethanol and filtered with suction, and the filter cake was washed with 1.5 L of ethanol. The combined ethanol was distilled under reduced pressure at 40-50° C. to a residue with 2 L of ethanol remaining, the temperature was lowered to 20-30° C., and 90 L of ethyl acetate was added with stirring for 2 h. The resulting mixture was filtered with suction, and the filter cake was washed with 4 L ethyl acetate, and dried under reduced pressure at 40-50° C. to constant weight, to give 2.4 kg of MN08 as a white solid with a yield of 65%.

Some embodiments and examples of the present invention are provided herein for the purpose of illustration, and they are not used to limit the scope of the invention. Some changes and modifications may be made based on common techniques in the field, but they should be construed to be within the scope of protection of the invention.

1. A process for manufacture of amantadine nitrate derivatives, comprising following steps: (1) synthesis of adamantanol from adamantane; (2) carboxylation of adamantanol; (3) acetylation of adamantanoic acid; (4) reduction; (5) hydrolysis of amido adamantanol and Boc protection of amino group; (6) crystallization of Boc protected amantadinol; (7) nitrate esterification of Boc protected amantadinol; (8) refining of the product of nitrate esterification; (9) Boc deprotection and salt formation; and (10) refining of amantadine nitrate hydrochloride: 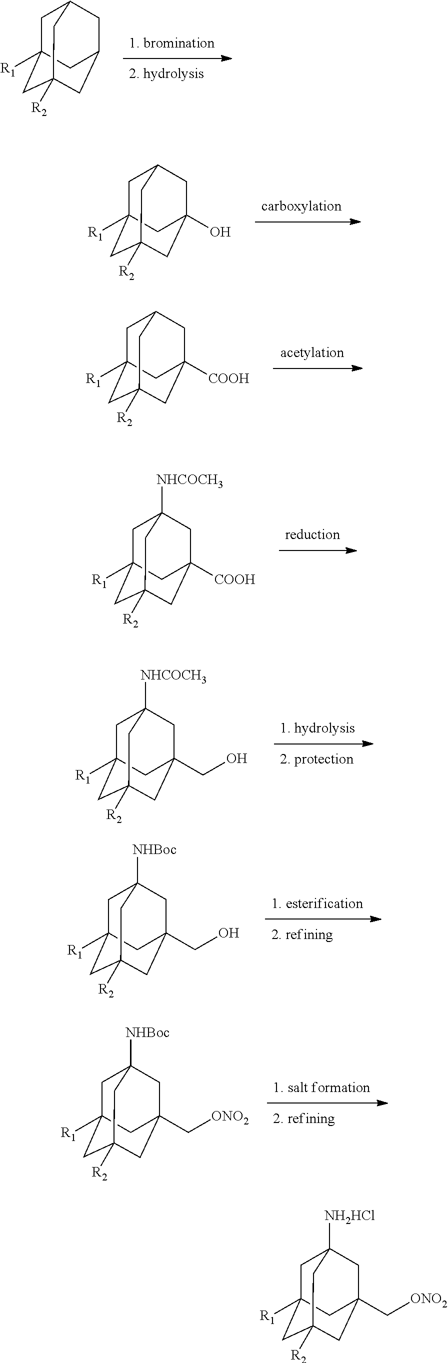
wherein, the adamantane has the structure of: 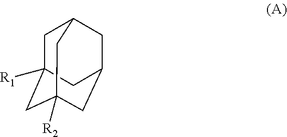
wherein, R₁ and R₂ are each independently hydrogen, straight-chain or branched-chain alkyl, substituted or unsubstituted aryl or heteroaryl;
wherein, the steps further comprise:
(1) synthesis of adamantanol: treating a substituted or unsubstituted adamantane to be brominated with liquid bromine and hydrolyzed with sodium oxalate to form an adamantanol;
(2) carboxylation of adamantanol: treating the adamantanol via Koch-Haff reaction, and condensation with formic acid to obtain an adamantanoic acid;
(3) acetylation of adamantanoic acid: treating the damantanoic acid via condensation with acetonitrile via Ritter reaction to obtain an amido adamantanic acid;
(4) reduction: treating the amido adamantanic acid via reduction with sodium borohydride to obtain an amido adamantanol;
(5) hydrolysis of amido adamantanol and Boc protection of amino group: treating the amido adamantanol, after an acyl group thereof being removed with strong alkali hydrolysis, via condensation with Boc anhydride;
(6) crystallization of Boc protected amantadinol: treating the product of Boc condensation via crystallization with mashing in n-hexane;
(7) nitrate esterification of Boc protected amantadinol: reacting the Boc protected amantadinol with a nitrating agent at low temperature to obtain a product of nitrate esterification, wherein the nitrating agent is a reagent with a mixture of fuming nitric acid and acetic anhydride;
(8) refining of the product of nitrate esterification: dissolving the product of nitrate esterification in alcohol, and adding water for crystallization with mashing;
(9) Boc deprotection and salt formation: removing Boc protecting group of the product obtained in step (8) via hydrolysis with HCl to obtain amantadine nitrate hydrochloride; and
(10) refining of amantadine nitrate hydrochloride: dissolving the amantadine nitrate hydrochloride in ethanol, resulting solution being filtered, concentrated and evaporated to remove partially the ethanol, adding ethyl acetate for crystallization with mashing, to obtain, after centrifugation, an amantadine nitrate hydrochloride;
wherein, in step (5), reaction solution for hydrolysis is polyethylene glycol; and in step (7), molar ratio of reaction feed is raw material:acetic anhydride:fuming nitric acid=1:1.08-1.8:1.68-2.8; and in step (9), molar ratio of reaction feed is raw material:HCl=1: 5-10.

2. (canceled)

3. The process according to claim 1, wherein step (1) further comprises:
(1a) Bromination: reacting a substituted or unsubstituted adamantane with liquid bromine in reflux for 4-6 h to obtain bromoadamantane; and
(1b) Hydrolysis: adding sodium oxalate and water, the reaction system being refluxed at 75° C. via hydrolization to obtain a substituted or unsubstituted adamantanol.

4. The process according to claim 1, wherein step (2) further comprises: the product from step (1) being dissolved in formic acid, and slowly added dropwise to concentrated sulfuric acid with cooling to 0-10° C., and then the reaction being maintained at 0-10° C. for 3-6 h; the reaction solution being poured into ice water and stirred for 16-18 h; after suction filtration, the solid filter cake being washed with water, dissolved with 0.27× sodium hydroxide solution, and filtered with suction; the filtrate being acidified with 5-10% hydrochloric acid to pH=1-2 and filtered, the filter cake being washed with water until the filtrate being neutral, and dried at 40-50° C. to obtain adamantanoic acid.

5. The process according to claim 1, wherein step (3) further comprises: cooling concentrated sulfuric acid to 0-10° C., and adding adamantanoic acid to be dissolved with stirring, adding slowly dropwise nitric acid, and then adding dropwise acetonitrile, reaction being maintained at 0-10° C. for 2-3 h; reaction solution being poured into ice water with stirring for 16-18 h, and filtered with suction, and filter cake being washed with water and dried at 40-50° C. to obtain amido adamantanic acid.

6. The process according to claim 1, wherein step (4) further comprises: adding the product of step (3) to tetrahydrofuran, cooled to 0-10° C., and adding triethylamine in batches, then adding dropwise ethyl chloroformate, and reaction being run at room temperature for 4-6 h; filtering resulting reaction mixture, and washing filter cake with tetrahydrofuran, combing tetrahydrofuran phases and cooling to 0-10° C., adding sodium borohydride in batches, then adding 0.8-1× water dropwise, and reaction being run for 2-3 h; adding 5× water, filtering reaction mixture, filtrate being spin-dried, then extracted with ethyl acetate, and combined ethyl acetate phases being dried over sodium sulfate, filtered, and spin dried, and residue being added with 1.5-2.5× ethyl acetate, stirred well, the same amount of petroleum ether being added, and stirred for 12-16 h, filtered and filter cake dried at 40-50° C. to obtain an amido adamantanol.

7. The process according to claim 1, wherein step (5) comprises following reactions:
(5a) Hydrolysis: adding amido adamantanol and a strong base sequentially to polyethylene glycol with a 5-10 times volume, reacted via high temperature hydrolysis at 100-180° C. for 10-18 h, and reaction system being cooled, diluted with purified water and stirred evenly, and water layer being extracted with toluene, dried over sodium sulfate and filtered, and the filtrate being concentrated; and
(5b) Condensation: dissolving the product obtained in reaction (5a) in dichloromethane, adding Boc anhydride, then running reaction via condensation at 20-30° C. for 1-5 h, and resulting mixture being concentrated to remove dichloromethane.

8. The process according to claim 1, wherein step (6) further comprises: adding n-Hexane to resulting residual oily liquid of step (5), reacted via crystallization for 2-3 h, and the resulting mixture being centrifuged, washed with n-hexane, and then dried at 40-50° C. for 16-18 h to obtain Boc-protected amantadinol.

9. The process according to claim 1, wherein step (7) further comprises: mixing fuming nitric acid with acetic anhydride at a temperature of −10° C. to 10° C. to give a nitrating reagent; dissolving resulting solid product from step (6) in dichloromethane with temperature controlled at −10° C. to 10° C., adding dropwise the nitrating reagent to be reacted for 15 min to 6 h, then quenching reaction upon its completion with ice water and water layer being separated, and organic layer being washed respectively with 10 times volume (10 Vol) of a solution of saturated sodium bicarbonate and a solution of saturated sodium chloride, dried over sodium sulfate, filtered, and concentrated to remove dichloromethane to obtain a crude product of Boc-protected amantadine nitrate.

10. The process according to claim 1, wherein step (8) further comprises: dissolving the crude product of step (7) in alcohol, adding water for crystallization with mashing for 2-3 h, then the resulting mixture being filtered, and resulting solid being dried at 40-50° C. to obtain a refined product.

11. The process according to claim 1, step (9) further comprises: dissolving the refined product of step (8) in ethyl acetate with temperature controlled at 10-30° C., adding HCl/ethyl acetate, running reaction for 16-18 h, and then resulting mixture being filtered with suction, and dried to obtain an amantadine nitrate hydrochloride.

12. The process according to claim 1, step (10) further comprises: adding product of step (9) in ethanol, treating via filtration with filtrate being concentrated, and adding ethyl acetate for crystallization with beating, resulting mixture being dried to constant weight to obtain a refined amantadine nitrate hydrochloride.

13. The process according to claim 3, wherein, in reaction of (1a) bromination of step (1), molar ratio of reaction feed is raw material:bromine=1: 2-6, reaction temperature is 60-90° C., and reaction time is 4 to 6 h; wherein, in reaction of (1b) hydrolysis of step (1), molar ratio of reaction feed is raw material:sodium oxalate=1:2.5-6.0, amount of water used is 23-35×, and reaction time is 2 to 3 h; wherein, in step (1) post-treatment process comprises: adding 3-5× sodium sulfite solution to reaction solution, stirring at room temperature for 16-18 h and then filtered, and filter cake being washed with water and then dissolved with 3-8× organic solvent, dried over anhydrous sodium sulfate, and filtered, and filter cake being washed with 2-4× organic solvent, and spin-dried under reduced pressure to obtain adamantanol as a solid.

14. The process according to claim 4, wherein, in step (2), molar ratio of reaction feed is raw material:formic acid:sulfuric acid=1: 6-10:11-16.

15. The process according to claim 1, wherein, in step (3), molar ratio of reaction feed is raw material:nitric acid:sulfuric acid:acetonitrile=1:1.6-2.5:9-15:1.1-1.5; wherein, in step (4), molar ratio of reaction feed is raw material:triethylamine:ethyl chloroformate:sodium borohydride=1:1.2-1.5:1.2-1.5:1.5-2.5.

16. The process according to claim 7, wherein, in the hydrolysis of step (5), the strong base is lithium hydroxide, sodium hydroxide or potassium hydroxide, and molar ratio of reaction feed is raw material:base=1: 5-10; wherein, reaction temperature is 100-180° C., reaction time is 15-16 h; wherein, in the condensation of step (5), amount of dichloromethane is 5-10 times in volume of raw material, and molar ratio of reaction feed is raw material:Boc anhydride=1:1.05-1.5, and reaction time is 1-5 h.

17. The process according to claim 8, wherein in step (6), the amount of n-hexane used for crystallization is 5-9 times in volume of raw material.

18. The process according to claim 9, wherein in step (7), molar ratio of reactants is raw material:acetic anhydride:fuming nitric acid=1:1.08-1.8:1.68-2.8; wherein the nitrating reagent is prepared at −10-10° C. and stirred for 0.5-1 h; and wherein reaction temperature is 0-10° C., and reaction time is 15 min to 6 h, or 30 min to 3 h.

19. The process according to claim 10, wherein in the step (8), the alcohol used for refining is methanol or ethanol, and weight ratio of reaction feed is raw material:alcohol:water=1: 2-5:3-8; wherein, after crude product of step (7) is completely dissolved in alcohol, a portion of the water was added, and stirred until precipitation of a white solid, then further stirred for 0.5-1 h, and remaining portion of the water was added with beating for 1-2 h; and wherein temperature for crystallization is controlled at 20-30° C.

20. The process according to claim 11, wherein, in step (9), concentration of HCl in HCl/ethyl acetate is in the range of 2.0-4.37 M, molar ratio of reaction feed is raw material:HCl=1: 5-10, reaction temperature is 20-30° C., and reaction time is 16-18 h; and wherein, refined product of step (8) is first dissolved in 3-6 times the weight of ethyl acetate, and then HCl/ethyl acetate is added for reaction.

21. The process according to claim 12, wherein, step (10) further comprising: dissolving the product of step (9) in alcohol, and concentrated under reduced pressure to remain 0.5-1 times of ethanol in the system, then adding ethyl acetate with mashing for 2-3 h, and wherein weight ratio of reaction feed is raw material:ethanol:ethyl acetate=1:2.5-3: 15-25, temperature of concentration with ethanol is 40-50° C., and temperature for drying is 45-50° C.
